(19) United States
(12) Patent Application Publication
(10) Pub. No.: US 2018/0042309 A1
(43) Pub. Date: Feb. 15, 2018

(54) Skin Accessible Cuff

(71) Applicant: William J. Brennan, Brentwood, TN (US)

(72) Inventor: William J. Brennan, Brentwood, TN (US)

(21) Appl. No.: 15/657,154

(22) Filed: Jul. 23, 2017

Related U.S. Application Data
(60) Provisional application No. 62/369,160, filed on Jul. 31, 2016.

(51) Int. Cl. A41B 7/00; A61B 5/00; A41D 27/28

(52) U.S. Cl. CPC A41B 7/00; A41D 27/28; A61B 5/681

(57) ABSTRACT

Openings in an industry standard long sleeved garment, form a portal to provide for the beneficial viewing of a wrist worn item and persistent contact with skin. The present invention is applicable to both standard long sleeve garments with attachable vents in the cuff area, found frequently on more formal garments, and is also applicable to industry standard long sleeve garments that are traditionally void of vented attachable openings in the cuff area, such as long sleeved T-shirts and long sleeved sweatshirts.

### DESCRIPTION OF FIELD

In the early 1900s, the collar and cuff of a shirt were often considered the only parts of a shirt that were socially acceptable to show in public. Due to the lack of ease and frequency of performing laundry functions, the collar and whole cuff on many long sleeved garments were frequently made removable so they could be changed or cleaned to look presentable. With the introduction of the washing machine, the need for removable collars and cuffs reduced in necessity. This brought about a long sleeved garment with a collar and a cuff no longer removable and more similar to what is traditionally worn today. Additionally, the invention and economic production of elastic materials, the requirement for a vent and fastener at the end of the cuff was also reduced as the elastic allowed for the opening in the cuff to dynamically expand and contract when penetrated by a hand. Cuffs containing elasticity are most commonly found on long sleeved T-shirts and long sleeved sweatshirts, while more formal long sleeved garments continue to commonly employ a vent and an attachable fastener.

### Engineering Constraints and Variables

In order to create an opening that allows a watch face or other wrist worn item to be consistently viewable while wearing a long sleeved garment, and simultaneously providing for any required consistent contact with skin, turned out to be significant engineering challenge. Although, in some ways, the final solution and embodiments appear simple, it was actually derived through a multitude of iterations and accommodate a wide range of variables. Consideration had to be given for the fact, not all long sleeved garments are the same type or size, garments are made from different material types, not all people wear their long sleeved garments the same way, not all wrist worn items are the same, the requirement for contact with skin varies, the size of a display area can vary greatly, etc. With all of these, and more, variables in mind, it became necessary to create a unique approach, to make it possible to consistently view a display area of a wrist worn item through a viewing portal in the cuff area, while simultaneously maintaining any required contact with skin. Additionally, consideration for the range of possibilities included, the possible distances between the wrist and the cuff resulting from personal preference or manufacturer styling. It also became necessary to ensure the display area would stay in the viewing portal with enough consistency to meet normal expectations under normal use. In some cases, it is necessary to add an additional opening or slit(s), in order to provide sufficient persistency of position and form retention of the viewing portal, as both viewability and restraint from interference with skin contact had to simultaneously be achieved, and thus could not benefit from the stabilizing effects of material under the display area, as employed by some of the prior art. Further, much of the other prior art could only achieve something approximating, by working separately from the standard cuff on a long sleeved garment.

### FIELD OF INVENTION AND ADVANTAGES

This invention relates to all forms and types of long sleeve garments, including those with and those without a traditional Cuff Vent. The increasing frequency of use, and reliance on, wrist worn items is increasing the need for quicker viewing and ease of access, to the wrist worn item's display area. When compared to short sleeved garments and garments that have the sleeves rolled up or otherwise retracted, long sleeved garments frequently inhibit the timely viewing of the wrist worn item's display area and require additional physical acts, effort and time, before the wrist worn item's display area can be viewed.

When people wear short sleeved shirts or long sleeved shirts with the sleeves rolled up, they are able to instantly access their wrist worn item without the viewing being inhibited by the cuff area traditionally found at the end of a long sleeved garment. Humans are known to become used to, and prefer predictable and repeatable easy effort when performing the same task. When unencumbered by a long sleeved garment, the easy access and instant gratification from viewing the display area of a wrist worn item frequently becomes the standard by which a user expects to obtain access to the wrist worn item's display area. Any restriction of this routine can result in, semi-aware, mild annoyance or frustration. In fact, a person inhibited by even a split second from the expected, is scientifically proven to be an agitation to the human brain, as an unexpected delay frequently requires the use of extra energy, either mental, physical, or both, but at minimum, the brain generally does not like a delay in the deliverance of gratification from that which has come to be expected.

As smart watches and other types of wrist worn items keep evolving and incorporating more functionality, the frequency of viewing will increase, and thereby, need for unencumbered and easy access will increase. This need will be compounded by the fact many of the current and future wrist worn items require contact with skin.

The present invention overcomes the ease of viewing and access limitations found on traditional long sleeved garments by repositioning and/or modifying the standard Cuff Vent, defined below with more detail, as a Repositioned Cuff Vent, or adds one or more openings to the cuff area of a long sleeved garment that does not have a traditional Cuff Vent, defined below with more detail, as a Viewable Ventless Cuff.

Traditional long sleeved garments containing a Cuff Vent, as frequently found on more formal garments, have the Cuff Vent in a position that is away from the top of the wrist and traditionally away from the direct view of the person wearing the garment, thus, these standard non-Repositioned Cuff Vents provide no benefit to aid in the beneficial viewing of a wrist worn item's display area. The repositioning of the traditional Cuff Vent to a new position on the cuff provides for multiple unique and beneficial results superior to, and unequaled by, the prior art. Specifically, these include:
1. The full and complete retention of the Cuff Vent's original purpose and benefits are maintained.
2. The repositioning of the Cuff Vent now provides for an efficient multi use, by allowing for the beneficial viewing of a wrist worn item's display area, retains Cuff Vents original functionality, and provides for contact with skin.
3. The average consumers' ease of use and familiarity with a traditional Cuff Vent is superior to the prior art's nontraditional design.
4. The alignment with, and leveraging of, the average garment manufacturer's expertise and tooling for Cuff Vents and multi-size openings, such as button holes and slits, provides a superior design and lower cost approach over the prior art.
5. The need for less material or fabric, than is required by the prior art, provides for additional cost benefits over the prior art.
6. The increasing requirement for the underside of the wrist worn items display area to retain direct contact with skin is a major advantage of the present invention over prior art with designs that inhibit direct contact with skin.

Further, many less formal garments not containing a traditional Cuff Vent, including many sweatshirts and long sleeved T-shirts, also inhibit the timely access and viewing of a wrist worn item's display area. The present invention also provides multiple embodiments that provide a similar beneficial solution for these types of garments.

### TERMS DEFINED AND DEFINITIONS

Cuff Vent—As traditionally and frequently found on more formal styles of long sleeved garments, a Cuff Vent is an attachable opening facilitating the penetration and exit of a wearer's hand through the end of a garment's sleeve when the attachable opening is in an unattached state. Post penetration, the attachable opening can be attached by a wide range of fasteners. This traditional attachable opening at the end of a long sleeved garment defines a Cuff Vent.

Repositioned Cuff Vent—A Repositioned Cuff Vent is defined as a Cuff Vent that retains its original functional purpose and is relocated from its customary and traditional position on a cuff to a new position forming a portal for the beneficial viewing of a wrist worn item's display area.

Repositioned Modified Cuff Vent—A Repositioned Modified Cuff Vent is (i) a Repositioned Cuff Vent (ii) where the shape or size of the Cuff Vent is noticeably different from a traditional Cuff Vent, and (iii) the above referenced difference in shape or size is sufficient to form a portal for the beneficial viewing of a wrist worn item's display area. When both conditions (i) through (iii) above are met, a Repositioned Modified Cuff Vent is defined.

Attachable Elongated Cuff Vent—An Attachable Elongated Cuff Vent is a (i) Repositioned Cuff Vent that incorporates (ii) a noticeable and distinct narrow or extended area of attachable material or fabric in proximity to the cuff, (iii) where said extended area has proportions similar to a peninsula, isthmus, strip, or the like, (iv) can be attached by a plethora of fastener types at one end, and (v) aids in the forming of a portal for the beneficial viewing of a wrist worn item's display area. When (i) through (v) above, are all simultaneously present, an Attachable Elongated Cuff Vent is defined.

Aligned Elongated Strip—An Aligned Elongated Strip aids in the beneficial position retention of a wrist worn item's display area. The elongated area or strip(s) portion of an Aligned Elongated Strip, can be similar in shape to those found in Attachable Elongated Cuff Vent embodiments, however the orientation of the strip(s) is a 90 degree rotation from those displayed in an Attachable Elongated Cuff Vent embodiment, so that the strip(s), in an Aligned Elongated Strip, aligns with the long length of a sleeve and a portion of the strip(s) can run above or below a portion of a wrist worn item, but only if the configuration does not interfere or inhibit the skin sensors of the wrist worn item. The strip(s) can be attachable or non-attachable. When the above conditions are met, an Aligned Elongated Strip is defined.

Ventless Cuff—A Ventless Cuff is the cuff of a traditional long sleeved garment that customarily does not have a Cuff Vent or any other type of attachable opening in the cuff. Long sleeved T-shirts and long sleeved sweatshirts are common examples of long sleeved garments with ventless cuffs. A Ventless Cuff is defined as a cuff on a long sleeved garment void of an attachable opening.

Viewable Ventless Cuff—A Viewable Ventless Cuff is defined as a Ventless Cuff with a non-attachable opening in the cuff, having sufficient size, shape and position to form a portal for the beneficial viewing of a wrist worn item's display area.

### OVERVIEW OF THE DRAWINGS

The drawings illustrate many of the possible embodiments of the present invention. As referenced in the Engineering Constraints and Variables section, part of the ingenuity of the present invention can be seen in is ability to provide a beneficial result across a very wide range of variables. In fact, the number of possible combinations and permutations amongst and between all of the variables quickly climbs into the 1000s. Creating a solution that could accommodate the largest number of variables, remain low cost, consumer friendly, and consistently provide a consistent result required multiple revisions and iterations. Some of the key variables that had to be considered, included variances amongst garment manufacturers, the type, dimensions and characteristics of the fabric or material used in the garment, the style and fit of the garment, the range of preferences likely desired by consumers, the number and types of wrist worn items, the range of possible dimensions, characteristics and materials used to produce wrist worn items, including the possible requirement for persistent contact with the skin.

In those cases where, the material/fabric type of the long sleeved garment, the stiffness or fit of the cuff, the dimensions or fit of the wrist worn item, the requirement for persistent and sufficient contact with skin, or the size of the Repositioned Cuff Vent, including any combination thereof, is found to be insufficient or inadequate to form a portal with sufficient consistent visible access, to a wrist worn item's display area and sufficient persistent contact with skin, then, in those cases, the selection of an alternative embodiments, both shown and not shown, can provide the desire result. Some of these selection options include, the adding of an additional opening in the cuff to provide increased stability between the cuff, the wrist, and the wristband. Other embodiments include, the adding of multiple additional openings, incorporation of a Viewable Ventless Cuff, modifications to the shape of the Cuff Vent, or the incorporation of an Attachable Elongated Cuff Vent, each of the above optionally employed when applicable, beneficial, or required, in order to provide a sufficiently consistent and stable viewing portal having persistency of form with edges effectively refrained from transgressing the display area of the wrist worn item. The drawings only display a range of possible embodiments, as the present invention has multiple permutations and combinations of possible embodiments.

### DRAWING GROUPINGS AND BASIC DESCRIPTION

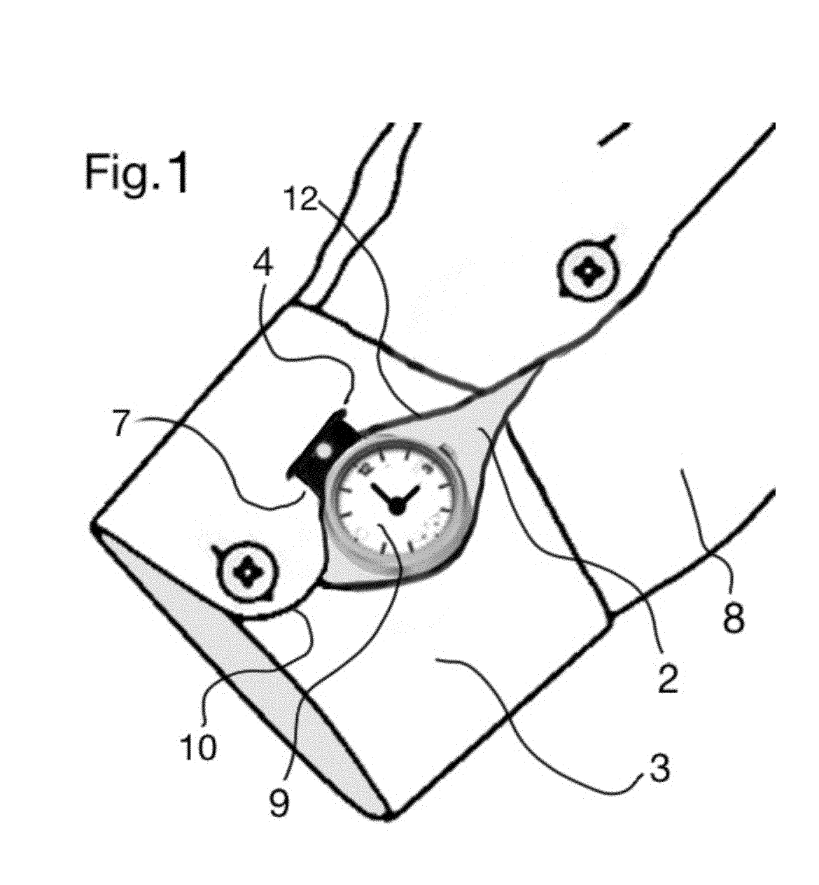

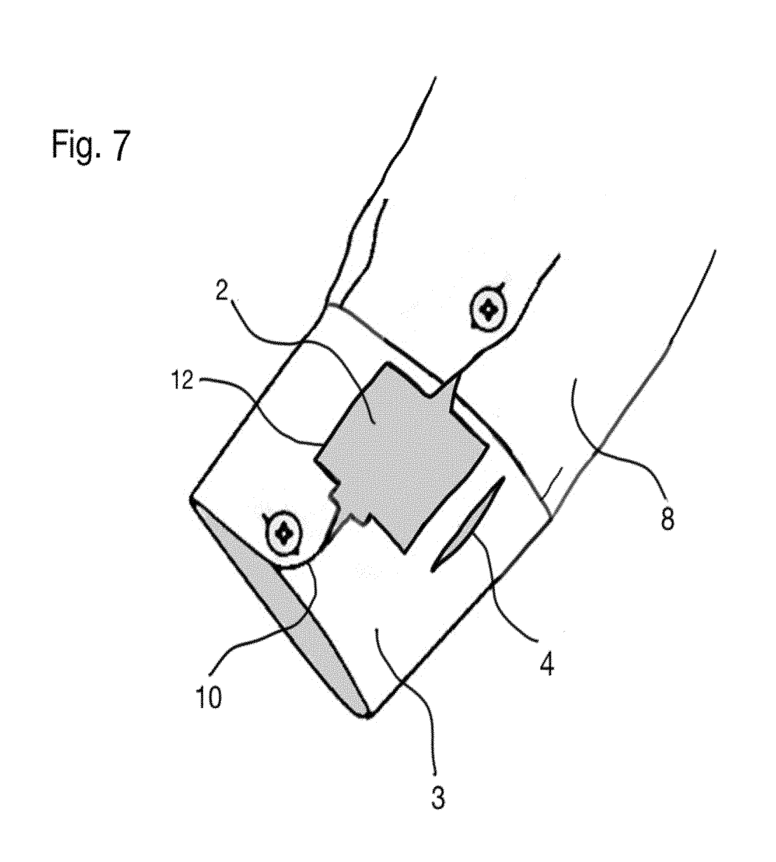

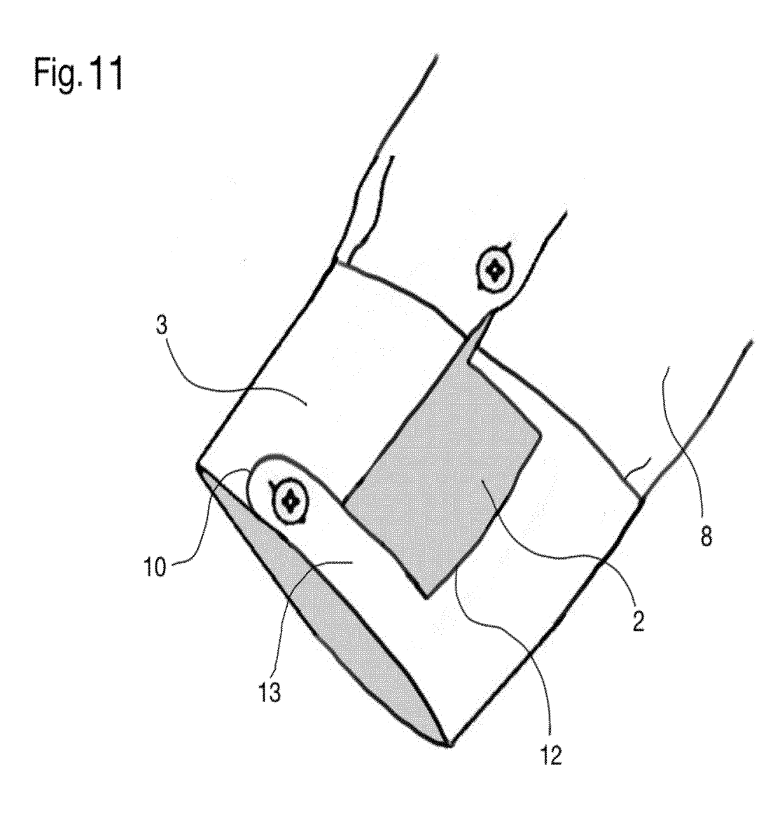

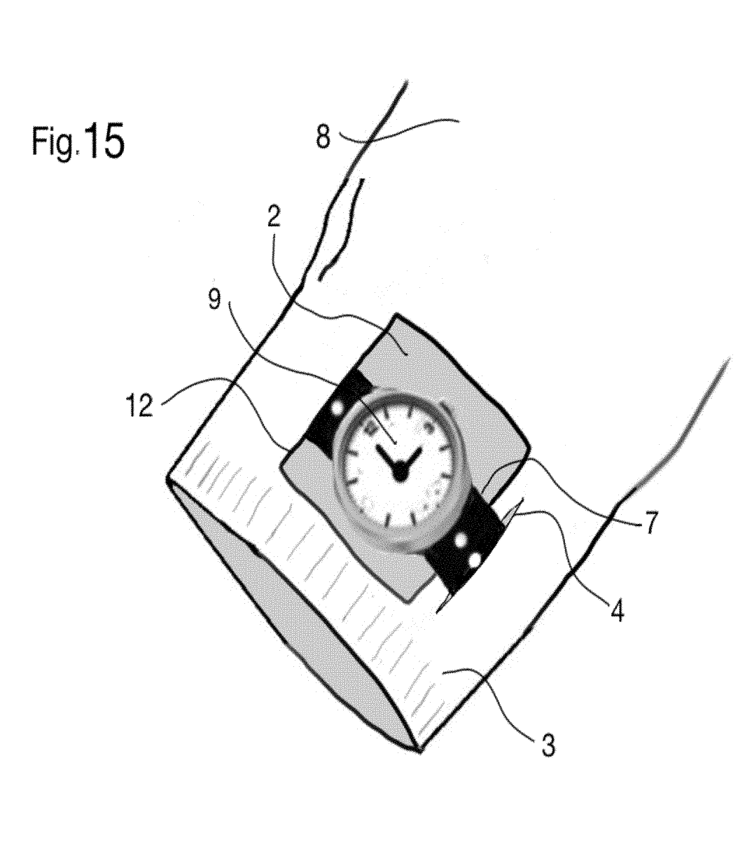

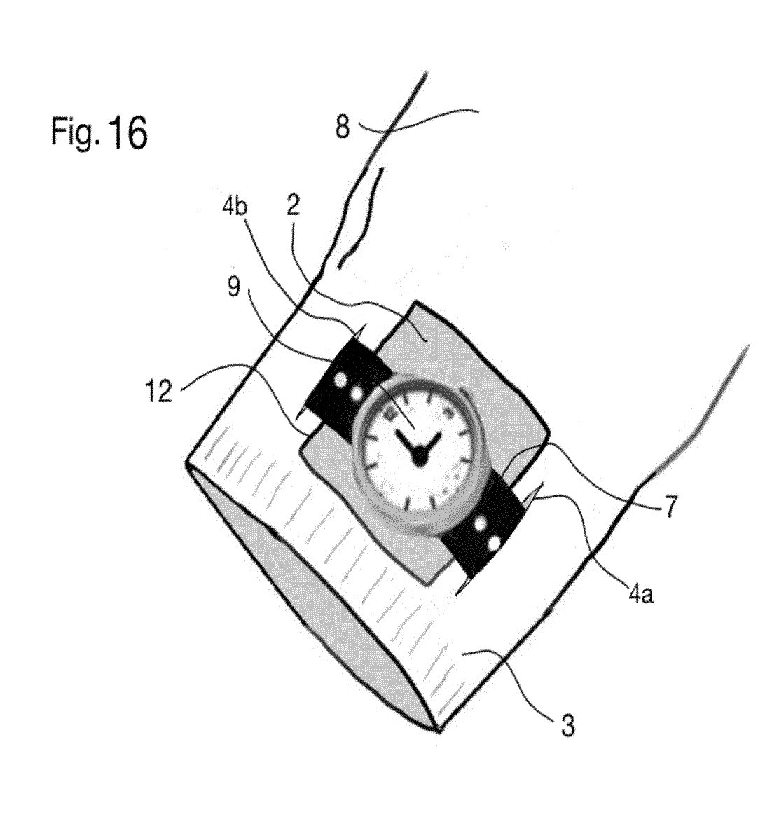

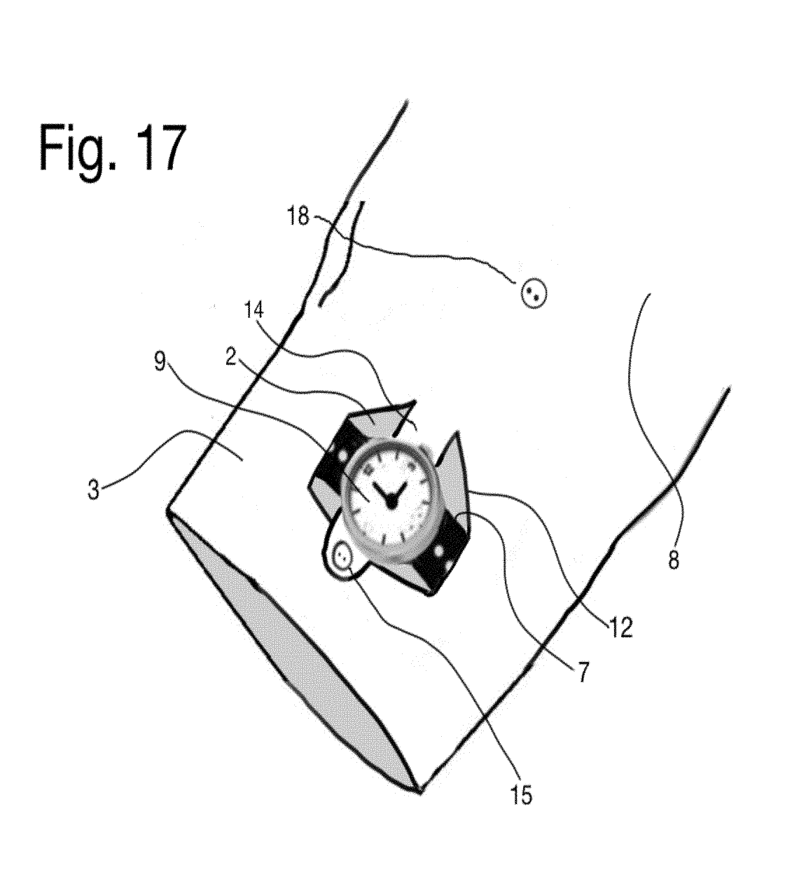

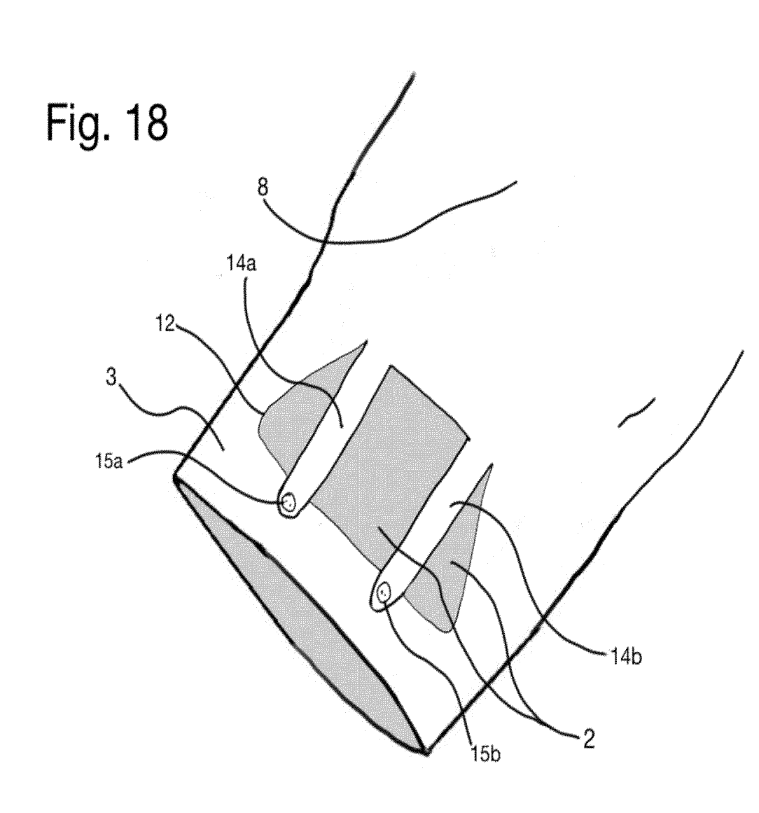

FIGS. 1-6 illustrate possible Repositioned Cuff Vent embodiments. FIGS. 7-10 illustrate possible Repositioned Modified Cuff Vent embodiments. FIGS. 11-14 illustrate possible Attachable Elongated Cuff Vent embodiments. FIGS. 15-16 illustrate possible Viewable Ventless Cuff embodiments. FIGS. 17-18 illustrate embodiments incorporating an Aligned Elongated Strip.

FIG. 1 shows a perspective view of an embodiment comprising a Repositioned Cuff Vent and one additional opening of the present invention. To aid in visualizing this embodiment of the present invention, an example of a wrist worn item is included in the drawing and is not part of the present invention.

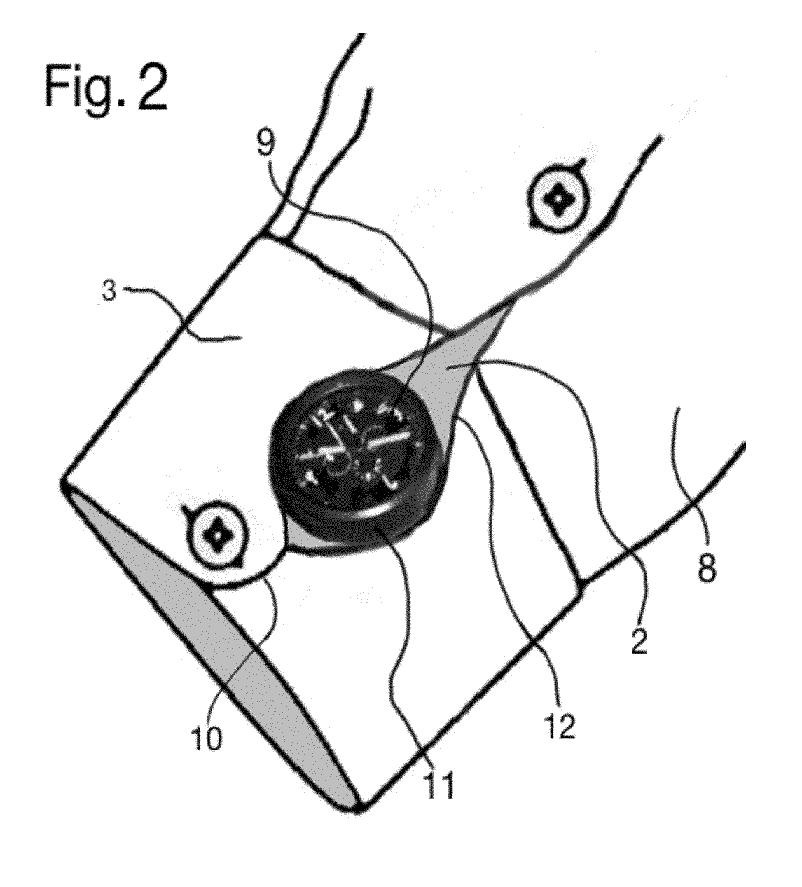

FIG. 2 shows a perspective view of an embodiment comprising a Repositioned Cuff Vent and no other openings of the present invention. To aid in visualizing this embodiment of the present invention, an example of a wrist worn item is included in the drawing and is not part of the present invention.

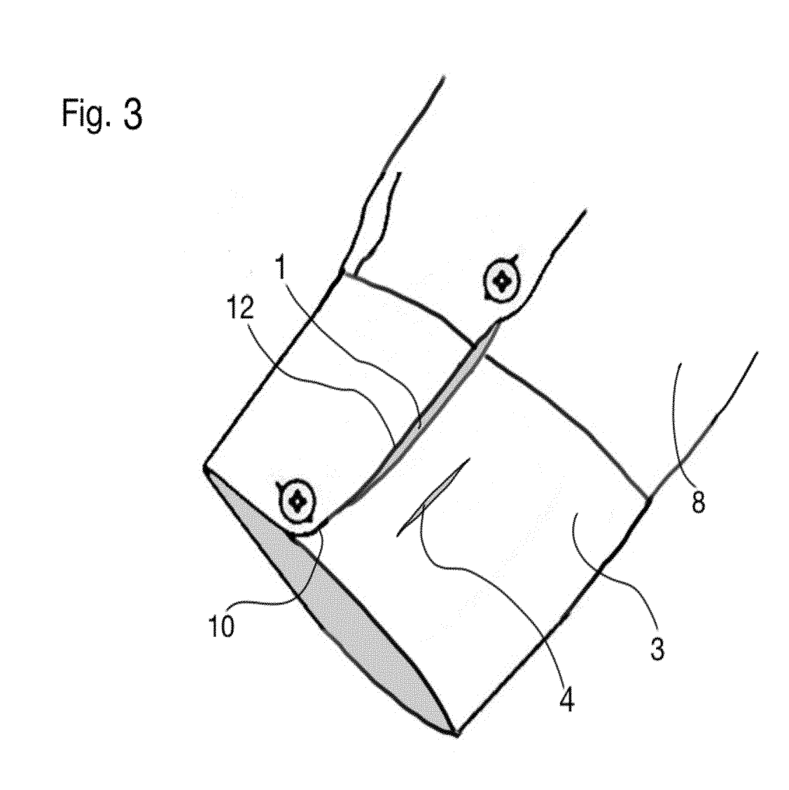

FIG. 3 shows an embodiment with a similar perspective view as FIG. 1, whereas the one additional opening of FIG. 1 is now located on the opposite side of the Repositioned Cuff Vent of the present invention, and the wrist worn item displayed in FIG. 1, is removed from FIG. 3.

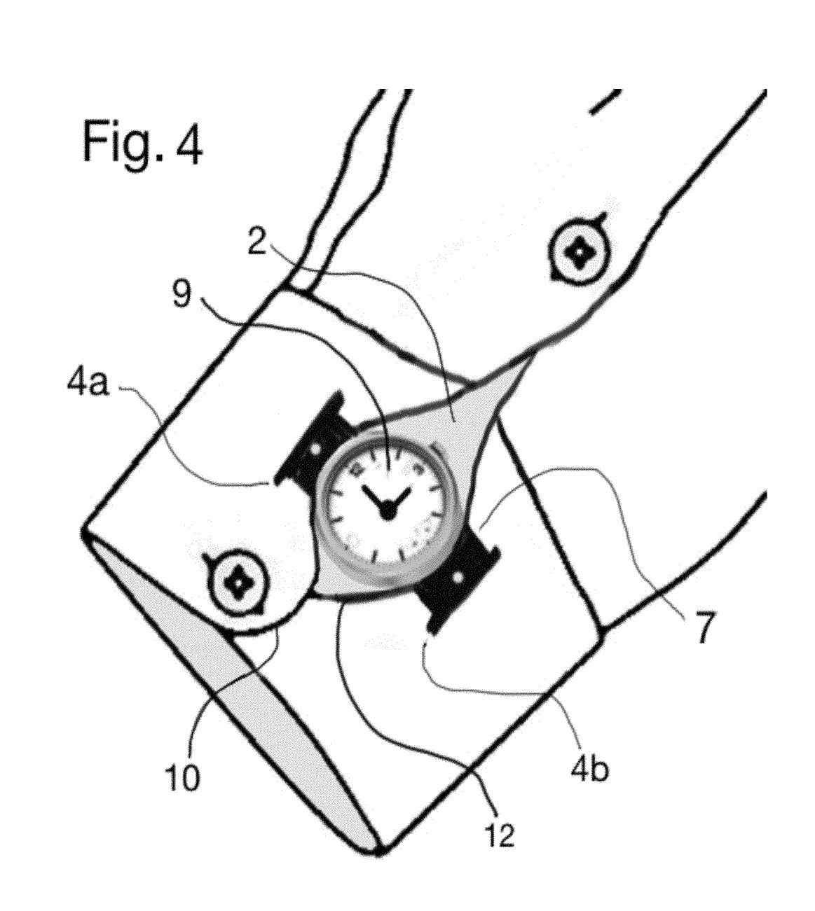

FIG. 4 shows an embodiment with a similar perspective view as FIG. 1, and adds a second additional opening in addition to the additional opening of FIG. 1. To aid in visualizing this embodiment of the present invention, an example of a wrist worn item is included in the drawing and is not part of the present invention.

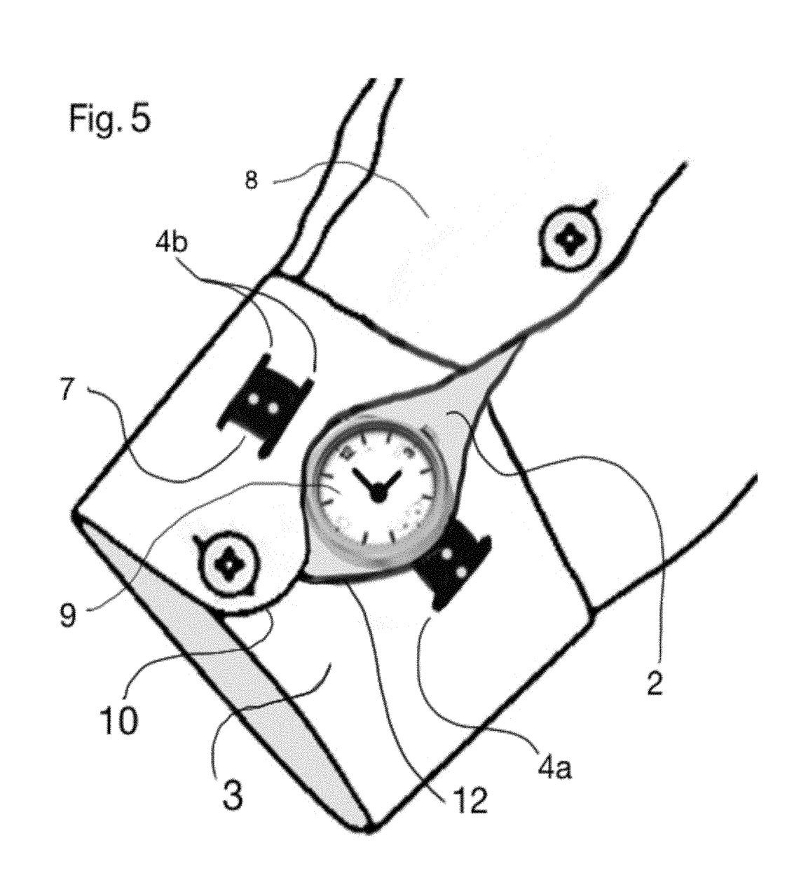

FIG. 5 shows an embodiment with a similar perspective view as FIG. 4, and adds an additional opening in addition to the openings present in FIG. 4. To aid in visualizing this embodiment of the present invention, an example of a wrist worn item is included in the drawing and is not part of the present invention.

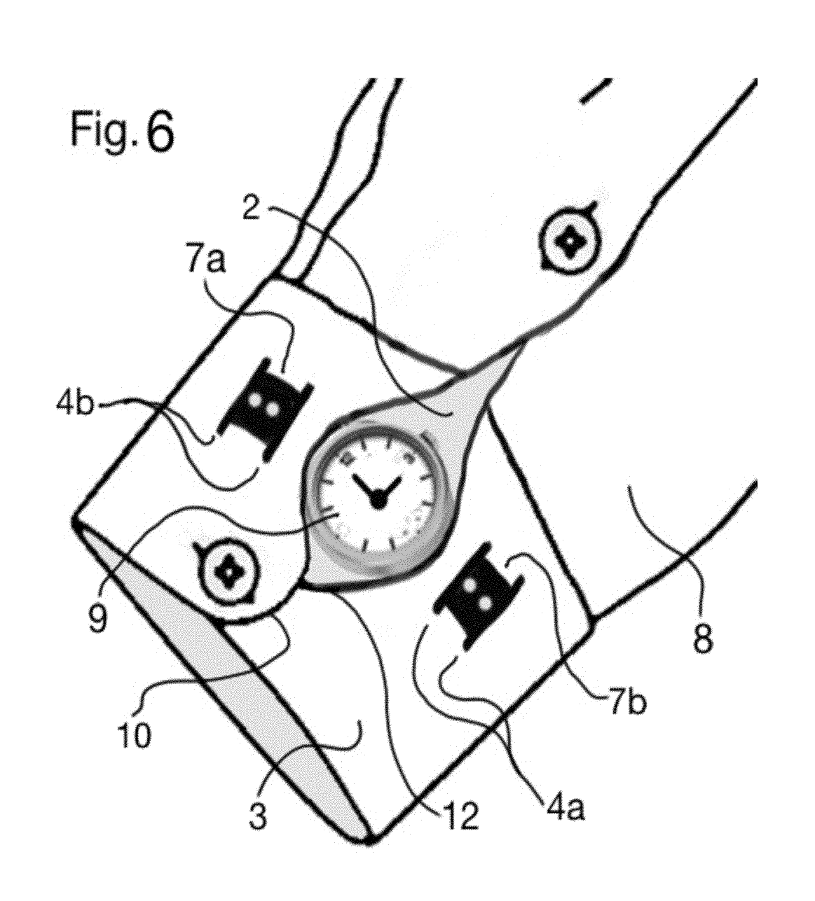

FIG. 6 shows an embodiment with a similar perspective view as FIG. 5, and adds an additional opening in addition to the openings present in FIG. 5. To aid in visualizing this embodiment of the present invention, an example of a wrist worn item is included in the drawing and is not part of the present invention.

FIG. 7 shows a perspective view of an embodiment comprising a Repositioned Modified Cuff Vent and one additional opening of the present invention.

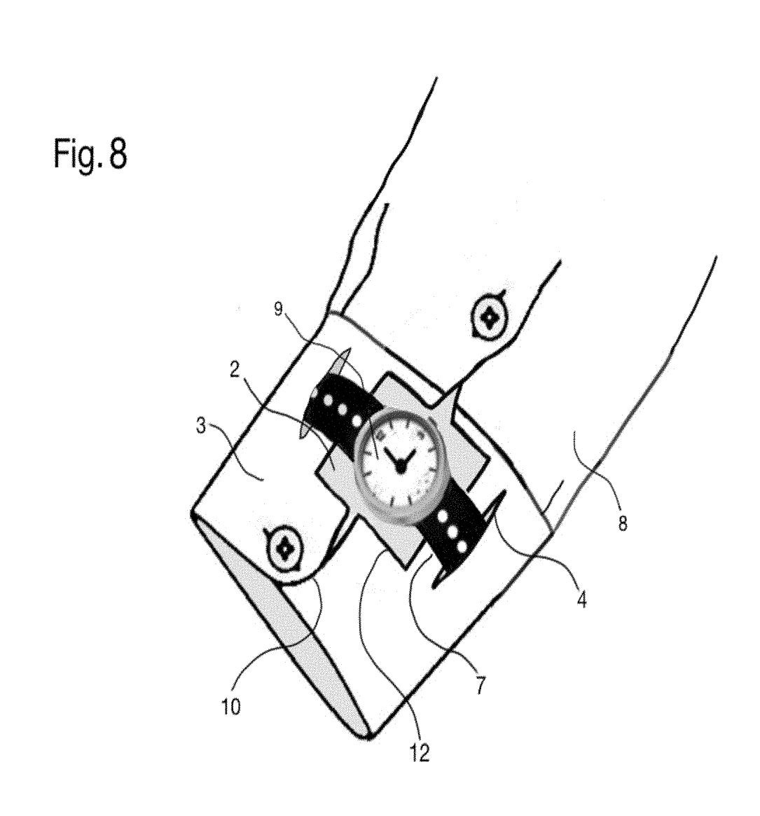

FIG. 8 shows a perspective view of an embodiment comprising a Repositioned Modified Cuff Vent having a slightly different shape than the shape in FIG. 7, and inclusion of two additional openings, of the present invention. To aid in visualizing this embodiment of the present invention, an example of a wrist worn item is included in the drawing and is not part of the present invention

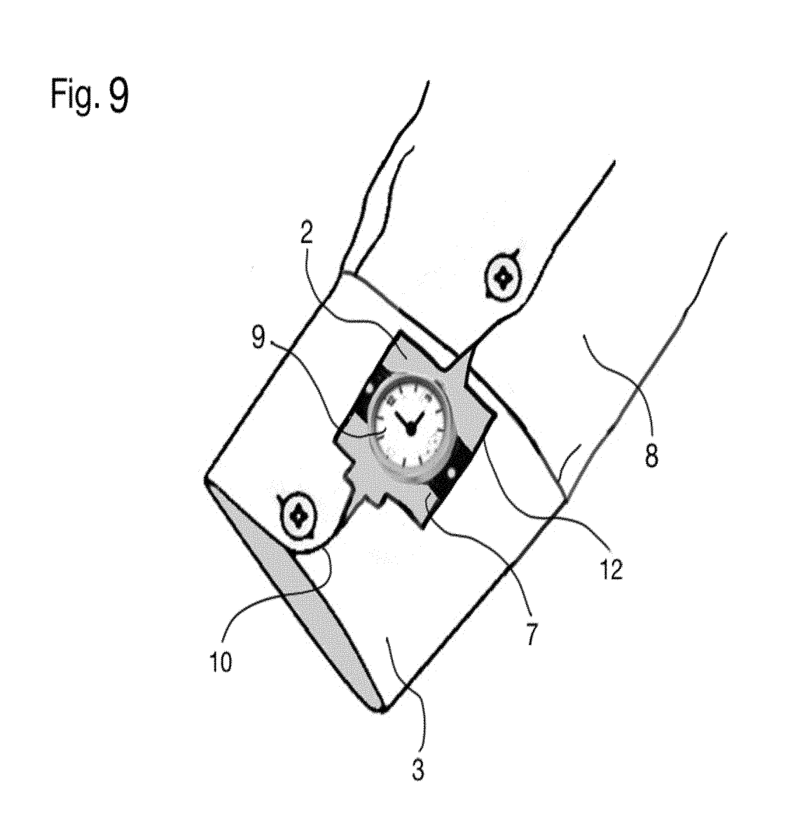

FIG. 9 shows a perspective view of an embodiment comprising a Repositioned Modified Cuff Vent and no additional openings of the present invention. To aid in visualizing this embodiment of the present invention, an example of a wrist worn item is included in the drawing and is not part of the present invention.

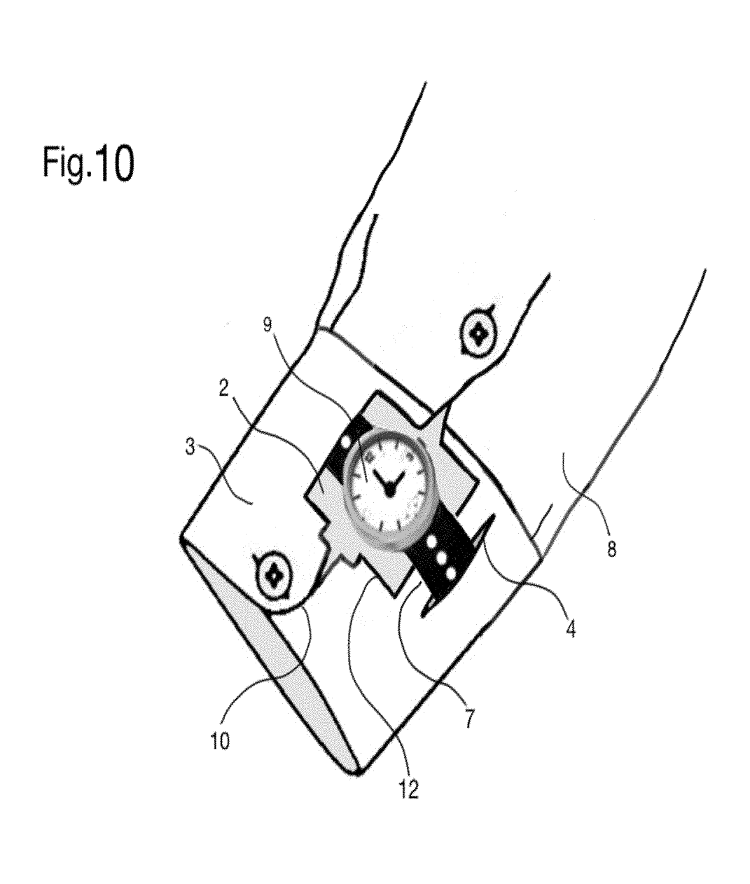

FIG. 10 shows an embodiment with a similar perspective view as FIG. 7 with the inclusion of a wrist worn item into the drawing to aid in visualizing this embodiment of the present invention. The wrist worn item is not a part of the present invention.

FIG. 11 shows a perspective view of an embodiment comprising an Attachable Elongated Cuff Vent and no other opening of the present invention.

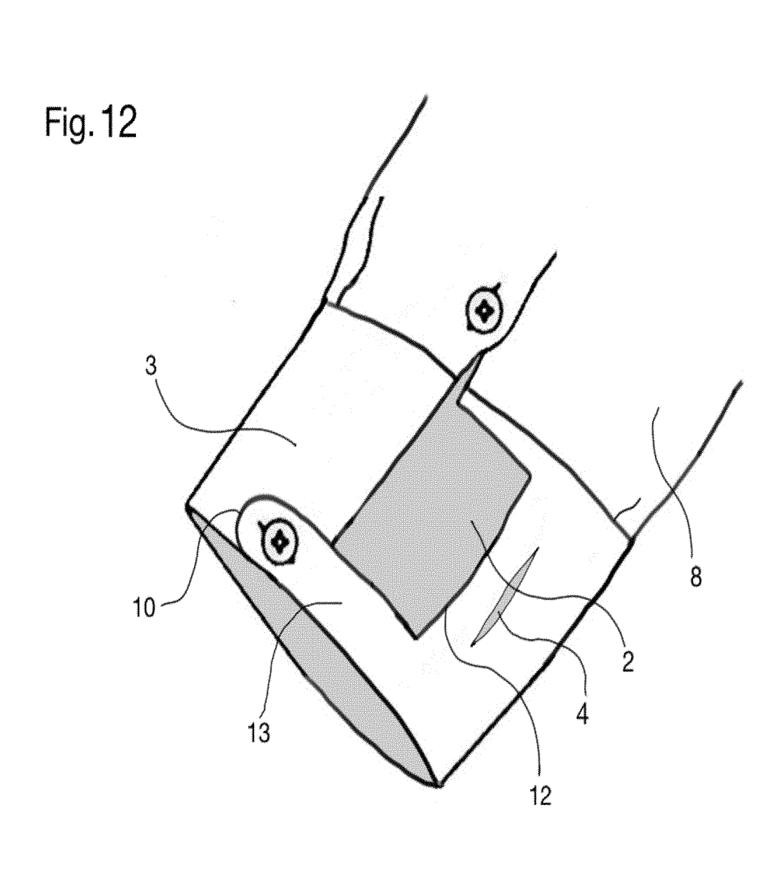

FIG. 12 shows a perspective view of an embodiment comprising an Attachable Elongated Cuff Vent and one additional opening of the present invention.

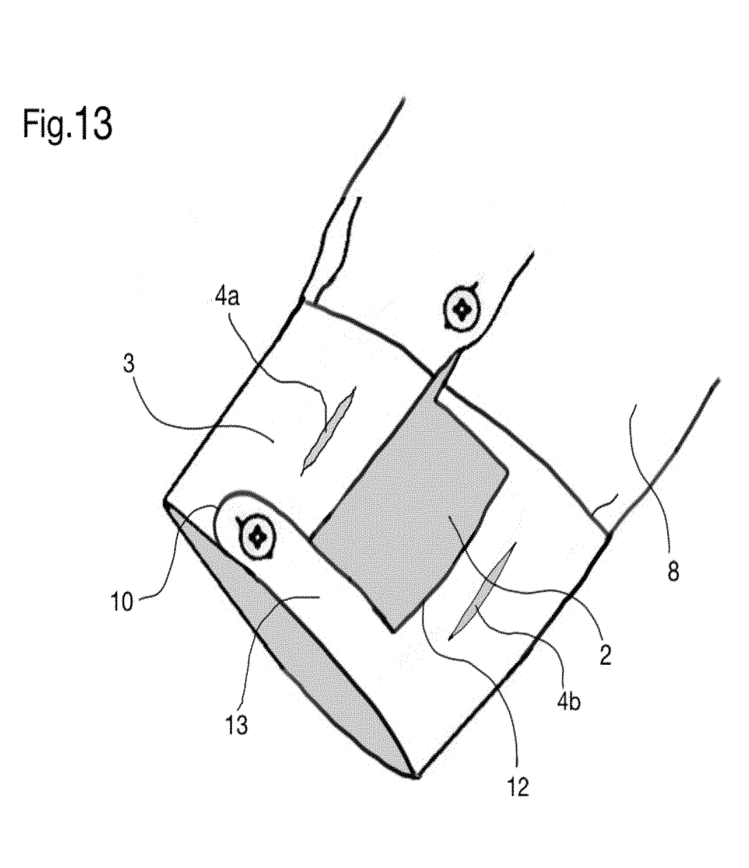

FIG. 13 shows a perspective view of an embodiment comprising an Attachable Elongated Cuff Vent and two additional openings of the present invention.

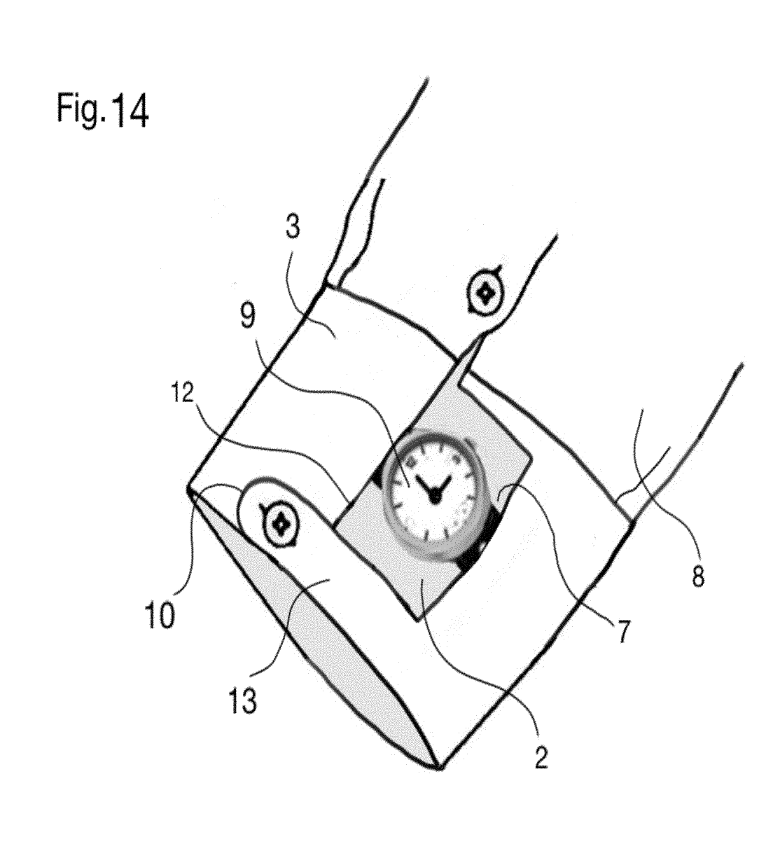

FIG. 14 shows an embodiment with a similar perspective view as FIG. 11, and to aid in visualizing this embodiment of the present invention, an example of a wrist worn item is included in the drawing and is not part of the present invention.

FIG. 15 shows a perspective view of an embodiment comprising a Viewable Ventless Cuff and one additional opening. To aid in visualizing this embodiment of the present invention, an example of a wrist worn item is included in the drawing and is not part of the present invention.

FIG. 16 shows a perspective view of an embodiment comprising a Viewable Ventless Cuff and two additional openings. To aid in visualizing this embodiment of the present invention, an example of a wrist worn item is included in the drawing and is not part of the present invention.

FIG. 17 shows a perspective view of an Aligned Elongated Strip embodiment with a single strip being displayed and the strip having a size and position sufficient to not interfere or inhibit the skin side sensors of compatible wrist worn items.

FIG. 18 shows a perspective view of an Aligned Elongated Strip embodiment with a two strips being displayed and the strips having size and position sufficient to not interfere or inhibit the skin side sensors of compatible wrist worn items.

### DETAILED DESCRIPTION OF THE DRAWINGS

FIG. 1 illustrates the first embodiment of a Repositioned Cuff Vent of the present invention. Comprising an industry standard long sleeved garment 8, a Repositioned Cuff Vent 10, and one additional opening 4 in cuff 3 where opening 4 can be penetrated by wristband 7 of a wrist worn item, to form a viewing portal 2. The edges 12 of the viewing portal 2 are sufficiently refrained from transgressing the display area 9 of a wrist worn item. Solely to aid in the visualization of this embodiment, an example of a wrist worn item is included in the drawing, without any intent or attempt to attach or claim the wrist worn item as a part of the present invention.

FIG. 2 illustrates an embodiment of present invention with a similar perspective view as FIG. 1 sans the additional opening labeled #4 in FIG. 1. This embodiment requires multiple independent variables to all be simultaneously present in order to produce a viable viewing portal. These variables include a long sleeved garment 8, with a Repositioned Cuff Vent 10, and the requirement for the cuff to retain relatively close contact with the skin 3 (snug fit), and a wrist worn item having a display area of sufficient profile height 11 or other means to retard the transgression of the viewing portal's edges 12 upon the display area 9 of a wrist worn item. Solely to aid in the visualization of this embodiment, an example of a wrist worn item is included in the drawing, without any intent or attempt to attach or claim the wrist worn item as a part of the present invention.

FIG. 3 illustrates a perspective view of an embodiment of present invention with similar aspects to FIG. 1, comprising a long sleeved garment 8 with a Repositioned Cuff Vent 10, and one additional opening 4, with this illustration displaying 4 in a different position as displayed in FIG. 1. The edges 12 of a viewing portal on cuff 3 is displayed prior to the wearing of a wrist worn item and prior to the expansion of the viewing portal 1.

FIG. 4 illustrates a perspective view of an embodiment of present invention with similar aspects to FIG. 1 with the main difference between the two illustrations, being the addition of one additional opening. The drawing comprises an industry standard long sleeved garment 8, a Repositioned Cuff Vent 10, and two additional openings 4a and 4b, in cuff 3 where opening 4a and 4b can both be penetrated by wristband 7 of a wrist worn item, to form a viewing portal 2. The edges 12 of the viewing portal 2 are sufficiently refrained from transgressing the display area 9 of a wrist worn item. Solely to aid in the visualization of this embodiment, an example of a wrist worn item is included in the drawing, without any intent or attempt to attach or claim the wrist worn item as a part of the present invention.

FIG. 5 illustrates a perspective view of an embodiment of present invention with similar aspects to FIG. 4 with the main difference between the two illustrations, being the addition of one additional opening. The drawing comprises an industry standard long sleeved garment 8, a Repositioned Cuff Vent 10, and three additional openings 4a and 4b, in cuff 3 where opening 4a and openings 4b can all be penetrated by wristband 7 of a wrist worn item, to form a viewing portal 2. The edges 12 of the viewing portal 2 are sufficiently refrained from transgressing the display area 9 of a wrist worn item. Solely to aid in the visualization of this embodiment, an example of a wrist worn item is included in the drawing, without any intent or attempt to attach or claim the wrist worn item as a part of the present invention.

FIG. 6 is the final illustration of a Repositioned Cuff Vent embodiment of the present invention and has a perspective view similar to FIG. 5 with the main difference between the two illustrations, being the addition of one additional opening. The drawing comprises an industry standard long sleeved garment 8, a Repositioned Cuff Vent 10, and four additional openings 4a and 4b, in cuff 3 where openings in 4a can be penetrated by wristband 7b and openings 4b can be penetrated by wristband 7a of a wrist worn item, to form a viewing portal 2. The edges 12 of the viewing portal 2 are sufficiently refrained from transgressing the display area 9 of a wrist worn item. Solely to aid in the visualization of this embodiment, an example of a wrist worn item is included in the drawing, without any intent or attempt to attach or claim the wrist worn item as a part of the present invention.

FIG. 7 is the first illustration of a Repositioned Modified Cuff Vent embodiment of present invention and displays one possible shape for a Repositioned Modified Cuff Vent. The drawing comprises a long sleeved garment 8, a Repositioned Modified Cuff Vent 10, one additional opening 4 in cuff 3, with edges 12 forming the boundaries of viewing portal 2.

FIG. 8 also illustrates a Repositioned Modified Cuff Vent embodiment similar to FIG. 7 with two of the main differences being one additional opening 4 being in addition to the additional opening in FIG. 7, and a slight difference in the shape of the Repositioned Modified Cuff Vent as compare to the one in FIG. 7, thereby displaying and confirming the present invention is not limited to any specific shape or dimensions as long as the resulting viewing portal results in the beneficial viewing of a wrist worn item's display area. The drawing is comprised of a long sleeved garment 8, a Repositioned Modified Cuff Vent 10, two additional openings in cuff 3 being penetrated by wristband 7 with edges 12 of the viewing portal 2 being sufficient for the beneficial viewing of the display area 9 of a wrist worn item. Solely to aid in the visualization of this embodiment, an example of a wrist worn item is included in the drawing, without any intent or attempt to attach or claim the wrist worn item as a part of the present invention.

FIG. 9 also illustrates a Repositioned Modified Cuff Vent embodiment similar to FIG. 7 sans the additional opening labeled as #4 in FIG. 7, and this illustration also includes a wrist worn item, which is not present in FIG. 7. This drawing displays a longed sleeved garment 8 and serves to demonstrate the present inventions ability to provide a viable viewing portal without need for opening(s) in cuff 3 to be penetrated by a wristband 7, when the supplied or created materials and configuration are such that the dimensions of the repositioned modified cuff 10, the distance of the viewing portal's edges 12 from the wrist worn item's display area 9, and the fit and stiffness of cuff 3, are collectively sufficient to maintain the size, position and shape of the viewing portal 2 with persistence over a wrist worn item's display area 9 without requiring additional openings. Solely to aid in the visualization of this embodiment, an example of a wrist worn item is included in the drawing, without any intent or attempt to attach or claim the wrist worn item as a part of the present invention.

FIG. 10 is the final illustration of a Repositioned Modified Cuff Vent similar to FIG. 8 sans one of the additional openings in shown in FIG. 8. The drawing is comprised of a long sleeved garment 8, a Repositioned Modified Cuff Vent 10, one additional opening in cuff 3 being penetrated by wristband 7 with edges 12 of the viewing portal 2 having sufficient proportions to provide for the beneficial viewing of the wrist worn item's display area 9. Solely to aid in the visualization of this embodiment, an example of a wrist worn item is included in the drawing, without any intent or attempt to attach or claim the wrist worn item as a part of the present invention.

FIG. 11 is the first illustration of an Attachable Elongated Cuff Vent embodiment of present invention. The visibly distinct elongated area of the Attachable Elongated Cuff Vent provides unique features and benefits over many of the other embodiments of the present invention, as it can simultaneously define the edge of the viewing portal, define the edge of the end of the cuff, provide the primary function of a Cuff Vent, and have no impact on one half of a standard Cuff Vent. The drawing displays a long sleeved garment 8, with a Repositioned Cuff Vent 10 in the form of an Attachable Elongated Cuff Vent 13 in cuff 3 with edges 12 forming the boundaries of the viewing portal 2.

FIG. 12 is the same illustration as FIG. 11 with one addition, that is, the addition of an additional opening 4 in cuff 3 to provide for increased position retention of the viewing portal 2.

FIG. 13 is the same illustration as FIG. 12 with one addition, that is the addition of an additional opening 4 in cuff 3 to provide for increased position retention of the viewing portal 2.

FIG. 14 illustrates the final drawing of the Attachable Elongated Cuff Vent embodiment of the present invention having a similar perspective view as FIG. 11, and some features similar to FIG. 9, by demonstrating the present inventions ability to provide a viable viewing portal without need for opening(s) in cuff 3 to be penetrated by a wristband 7, when the supplied or created materials and configuration are such that the dimensions of the repositioned modified cuff 10, the distance of the viewing portal's edges 12 from the wrist worn item's display area 9, and the fit and stiffness of the Attachable Elongated Cuff Vent 13 and cuff 3, are collectively sufficient to maintain the size, position and shape of the viewing portal 2 with persistence over a wrist worn item's display area 9 without requiring additional openings. Solely to aid in the visualization of this embodiment, an example of a wrist worn item is included in the drawing, without any intent or attempt to attach or claim the wrist worn item as a part of the present invention.

FIG. 15 illustrates the first drawing of the Viewable Ventless Cuff embodiment of the present invention. A Ventless Cuff is typically found on T-shirts, sweatshirts and the like, as long sleeved garments are traditionally ventless. The Viewable Ventless Cuff embodiment is beneficial when there is no Cuff Vent to reposition, and frequently this embodiment is the most effective choice to overcome the common inherent deficiencies of material quality often encountered in the cuff area for these types of long sleeved garments. The drawing displays a long sleeved garment 8 incorporating a Viewable Ventless Cuff having boundaries defined by the edges 12 of the viewing portal 2 in cuff 3 and incorporating one additional opening 4 to allow penetration of wristband 7 to provide for the beneficial viewing of a wrist worn item's display area 9. Solely to aid in the visualization of this embodiment, an example of a wrist worn item is included in the drawing, without any intent or attempt to attach or claim the wrist worn item as a part of the present invention.

FIG. 16 illustrates the last drawing of the Viewable Ventless Cuff embodiment of the present invention. The drawing is substantially similar to FIG. 15 except for the addition of one additional opening as compared to FIG. 15. The drawing displays a long sleeved garment 8 incorporating a Viewable Ventless Cuff having boundaries defined by the edges 12 of the viewing portal 2 in cuff 3 and incorporating two additional openings 4a and 4b to allow penetration by wristband 7 in multiple location of cuff 3 to provide, when required, superior position persistence and stability of viewing portal 2, as compared to FIG. 15 and its one less additional opening. When required, the additional opening can ensure the beneficial viewing of a wrist worn item's display area 9. Solely to aid in the visualization of this embodiment, an example of a wrist worn item is included in the drawing, without any intent or attempt to attach or claim the wrist worn item as a part of the present invention.

FIG. 17 illustrates an example of an Aligned Elongated Strip embodiment of present invention displaying a long sleeved garment 8 with a single elongated strip 14 displayed in one of its possible attached positions 15 and elongated strip 14 is displayed as residing partially below the wrist worn item's display area 9, to aid in position retention inside of viewing portal 2 defined by its edges 12 in cuff 3. Additional embodiments are possible when the position, size, shape or material type of elongated strip 14 is sufficient to allow for the proper functioning of a wrist worn item's sensors. Further, other embodiments of this same perspective could optionally display the elongated strip 14 with a position above a wrist worn item. It is also possible to reposition elongates strip 14 to an attached position 18 thereby removing all contact with a wrist worn item's display area 9 or its wristband 7. Finally, elongated strip 14 can also be embodied as non-attachable. Solely to aid in the visualization of the displayed embodiment and the referenced embodiments not displayed, an example of a wrist worn item is included in the drawing, without any intent or attempt to attach or claim the wrist worn item as a part of the present invention.

FIG. 18, also illustrates an additional embodiment of an Aligned Elongated Strip displaying two elongated strips 14a and 14b aiding in position retention of a wrist worn item's display area 9 inside viewing portal 2 as defined by its edges 12 in cuff 3 on a long sleeved garment 8, where the size, position, or material type of 14a and 14b, are all sufficient for the consistent beneficial viewing of display area 9 and persistent skin contact with the underside of display area 9 or wristband 7. Finally, the present invention provides for embodiment that are not displayed, such as 14a and 14b can be attachable or non-attachable at position 15a and 15b.

I claim:

1. A long-sleeved garment containing a repositioned cuff vent and at least one additional adjacent opening facilitating the beneficial viewing of a wrist worn item's display area.

2. A garment according to claim 1, wherein one or more additional openings are in proximity to the first additional opening.

3. A garment according to claim 1, wherein the first additional opening and all subsequent additional openings are of sufficient size and position to provide for the penetration of a wristband of a wrist worn item.

4. A garment according to claim 1, wherein the first opening is of sufficient size and position to provide for the beneficial viewing of a wrist worn item's display area.

5. A garment according to claim 1, wherein the first additional opening and all subsequent additional openings are a slit, hole, button hole, or the like.

6. A garment according to claim 1, wherein the first opening allows for a wrist worn item's persistent beneficial contact with skin.

7. A garment according to claim 1, wherein one or more additional openings facilitates the wrist worn item's persistent beneficial contact with skin.

8. A long-sleeved garment comprising an opening in the sleeve's cuff where the position, size and shape of the opening simultaneously facilitates both the beneficial viewing of a wrist worn item's display area and the beneficial persistent skin contact by the wrist worn item.

9. A garment according to claim 8, wherein one or more additional openings are in proximity to the opening.

10. A garment according to claim 8, wherein the opening is in a ventless cuff garment.

11. A garment according to claim 8, wherein the opening is in the cuff of a long-sleeved T-shirt or long-sleeved sweatshirt and the opening is not attachable.

12. A garment according to claim 8, wherein the opening is a repositioned cuff vent.

13. A garment according to claim 8, wherein the opening is a repositioned modified cuff vent.

14. A garment according to claim 8, wherein the opening is the absence of material traditionally present in a cuff.

15. A garment according to claim 8, wherein the opening facilitates the wrist worn item's persistent beneficial contact with skin.

16. A garment according to claim 8, wherein one or more additional openings facilitate the wrist worn item's persistent beneficial contact with skin.

17. A long-sleeved garment comprising an opening in the cuff area where at least one part of the opening is formed by an attachable elongated strip having size, shape, and rigidity, sufficient to simultaneously maintain the position of the opening over the wrist worn item's display area and allowing for the proper functioning of a wrist worn item's sensors.

18. A garment according to claim 17, where two or more elongated areas are present.

19. A garment according to claim 17, wherein one end of the elongated area can be attached by a fastener.

20. A garment according to claim 17, wherein one end of the elongated area is attached by a fastener and the fastener is a button, Velcro, snap, hook, cufflink, or the like.

21. A garment according to claim 17, wherein the opening facilitates the wrist worn item's persistent contact with skin.

22. A garment according to claim 17, wherein one or more additional openings in proximity to the opening facilitates the wrist worn item's persistent contact with skin.

23. A garment according to claim 17, wherein the elongated area is a strap, extension, strip, peninsula, protrusion, isthmus, or the like.

24. A garment according to claim 17, wherein the position, size, shape, or material type, are sufficient to provide a stable portal and a improved chance of recovery.
